(19) United States
(12) Patent Application Publication
(10) Pub. No.: US 2008/0154146 A1
(43) Pub. Date: Jun. 26, 2008

(54) System and method for managing neurosensory test information

(76) Inventors: A. L. Dellon, Baltimore, MD (US); Lajoo Motwani, Tucson, AZ (US)

Correspondence Address: PATTON BOGGS, LLP, 2001 ROSS AVENUE, SUITE 3000, DALLAS, TX, 75201, US

(21) Appl. No.: 11/643,281

(22) Filed: Dec. 21, 2006

(51) Int. Cl. A61B 5/00

(52) U.S. Cl. 600/544

(57) ABSTRACT

A device for determining the sensitivity of nerve function includes a first flexible beam and a second flexible beam. The second flexible beam is disposed outwardly from and is substantially parallel to the first flexible beam. The device also includes an interconnect in contact with the first flexible beam and the second flexible beam, wherein the first flexible beam is operable to be flexed in response to a load applied to the flexible beam.

### BACKGROUND OF THE INVENTION

Neurosensory injuries and surgical procedures to extremities, such as hands and feet; are a challenge to medical professionals in determining initial damage to nerves and recovery progress of the nerves. One way for diagnosing initial nerve damage and nerve recovery is to apply stimulation to points on a hand, such as fingers or palm, or foot, such as toes or sole. The stimulation that is typically applied for testing nerve function includes pressure, temperature, and/or electrical current, for example. One stimulation test that is often performed includes applying stimulation, such as pressure, from two nearby points. The purpose of the test is to determine the innervation density of the patient's fiber-receptor system in the area being tested.

There are generally two tests for determining innervation density, including dynamic and static testing. A dynamic pressure test (i.e., moving two points along the surface of the skin) assesses response of the quickly adapting fiber-receptor system. Dynamic tests are typically used to determine neurosensory functions requiring moving touch, such as object identification (e.g., buttoning a button). A static pressure test is typically used for determining neurosensory functions requiring pressure sensing, such as shaking a hand.

Another test that is often used includes a two-point discrimination test. A two-point discrimination test is performed by pressing two points against a portion of a person's skin and determining whether the person can sense both points. The two-point discrimination test is used for testing the slowly adapting fiber-receptor system. This is a static test and can be used to assess hand functions requiring a sensory grip and constant touch, such as holding tools, pencils or the like.

It has been well known to provide one or two-point discrimination tests. One device known as the DISK-CRIMINATOR® has been advanced, and it includes an octagonal disk that has a series of metal rods or prongs protruding from the periphery at different spacings (e.g., 2 mm-8 mm and 9 mm-16 mm). In operation, a patient may press one or two adjacent rods or prongs onto a test point for two-point discrimination testing. The use of such a device for testing is imprecise and subjective as the test giver is generally a medical professional or patient who is self-administering the test who “estimates” the amount of pressure exerted on the patient's skin. Therefore, there is a need for a more precise and less subjective two-point discriminating testing device to enable medical professionals and patients determine the healing progress of neurosensory injuries.

### SUMMARY

In an embodiment of the present invention, a load sensing cell is disclosed that includes a pair of substantially planar walls that are in substantially parallel relation to each other. The load sensing cell also includes a pair of interconnect side walls, each of which is connected to and extends between the pair of substantially planar walls. The load sensing cell further includes a strain gauge connected to at least one of the pair of planar walls in a manner that produces signals related to bending of the substantially planar walls in directions transverse to their planes. The load sensing cell is configured such that bending loads may be applied to the load sensing cell in directions substantially transverse to the planes of the substantially planar walls.

In another embodiment of the present invention, a device for determining nerve function response is disclosed that includes a first flexible beam and a second flexible beam. The second flexible beam is disposed outwardly from and is substantially parallel to the first flexible beam. The device also includes an interconnect in contact with the first flexible beam and the second flexible beam, wherein the first flexible beam is operable to be flexed in response to a load applied to the flexible beam.

In yet another embodiment of the present invention, a sensing device with a dual beam structure is disclosed for sensing human nerve function.

In a further embodiment of the present invention, a method of determining nerve function response is disclosed that includes measuring a flexure of a dual beam, the degree of flexure being related to nerve function response.

In an additional embodiment of the present invention, a device for determining nerve function response is disclosed that includes a dual beam and at least one sensor disposed on the dual beam operable to detect the flexure of the dual beam. The device also includes a processor operable to convert first data related to the degree of flexure of the dual beam into second data related nerve function response, such as sensibility.

Another embodiment may include a system for managing neurosensory test information. The system may include a neurosensory test apparatus configured to make at least one neurosensory test data reading from a patient. A computing system may operate on a network and be in communication with a storage unit. A data repository may be stored in the storage unit and be configured to store neurosensory test data read by the neurosensory test apparatus. Means for communicating the neurosensory test data to the data repository may be utilized.

Still yet, the principles of the present invention may provide for an apparatus for testing a neurosensory response from a patient. The apparatus may include a handheld computing device including a user interface, sensing electronics electrically connected to the handheld computing device, a housing configured to house the sensing electronics and support the handheld computing device, where the housing may further be configured to enable a user to access the user interface of the handheld computing device, and a neurosensory sensory device, operable to test a neurosensory response from a patient, in communication with the handheld computing device.

### BRIEF DESCRIPTION OF THE DRAWINGS

FIG. 1 is an illustration of one embodiment of a dual beam structure for determining nerve function response that is implemented according to the teachings of the present invention;

FIG. 2 is an illustration of one embodiment of a sensory device implemented according to the teachings of the present invention that utilizes a dual beam structure;

FIG. 3 is an illustration of another view of an embodiment of a sensory device implemented according to the teachings of the present invention that utilizes a dual beam structure;

FIG. 4 is an illustration of one embodiment of a circuit implemented according to the teachings of the present invention that detects changes in a beam such as the flexing or bending of such beam;

FIG. 5 is a flow diagram of an exemplary process for determining nerve function response according to the teachings of the present invention;

FIG. 6 is a block diagram of an exemplary configuration of a software architecture for collecting and storing neurosensory data in accordance with the principles of the present invention;

FIG. 7 is an illustration of an exemplary system configuration for measuring, storing, and accessing neurosensory test data;

FIG. 8 is a flow diagram of an exemplary process for measuring and storing neurosensory test data;

FIG. 9 is an illustration of an exploded view of an exemplary housing for enclosing a handheld computing device, such as a PDA, for collecting data from a sensory device used to perform neurosensory testing on patients;

FIG. 10 is an illustration of the exemplary housing of FIG. 9 in a working configuration; and

FIG. 11 is a flow diagram of an exemplary process for configuring an apparatus for testing a neurosensory response from a patient.

### DETAILED DESCRIPTION OF THE DRAWINGS

The present invention relates to an apparatus used to sense human patient nerve function. In one embodiment, the apparatus is portable for ease of transport and use between test centers and other locations. Reference to a “test center” means a hospital, doctor's office, rehabilitation facility, clinic or other facility or organization that will test patients, particularly for nerve health. In addition, a device being “portable” means that the device is easily moveable from a test center to another location, and easily moveable within the test center from one patient to another.

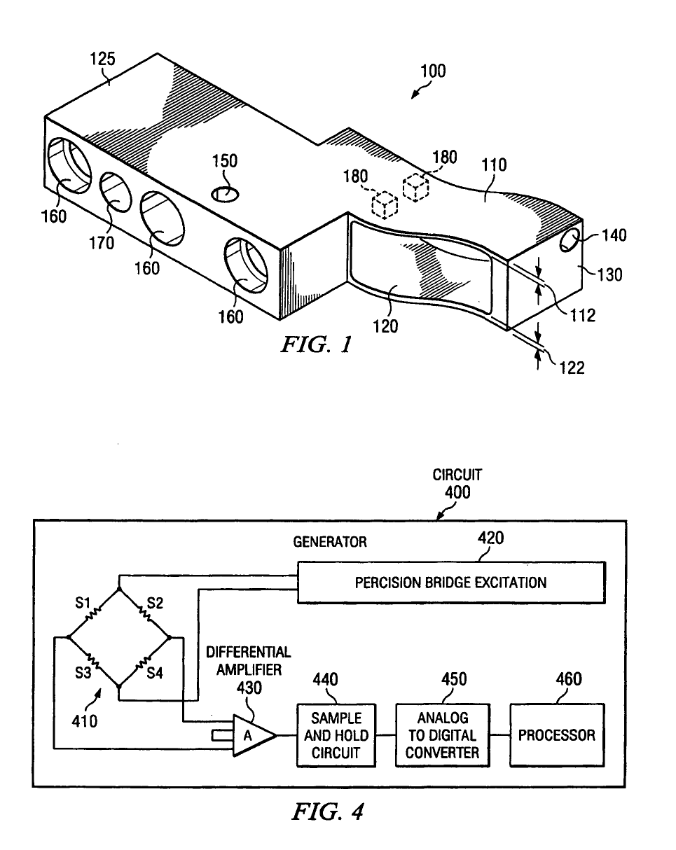

FIG. 1 illustrates one embodiment of a dual beam structure 100 useful for determining nerve function response that is implemented according to the teachings of the present invention. More particularly, dual beam structure 100 allows the use of beams of decreased thickness as compared to beams previously used in single beam structures such as the one described in U.S. Pat. No. 5,027,828. Such decreased thickness allows for the flexing of such beams in response to lower forces or pressure applied to such beams. In such a manner, a device for determining nerve function response that utilizes such dual beam structure 100 may have an enhanced ability to detect the sensibility of nerve function relative to smaller forces of stimulus to human tissue.

More particularly, dual beam structure 100 includes a beam 110 and a beam 120. Beam 110 and 120 are connected by interconnect 130. Beam 110 and beam 120, although described as separate beams, may be separate structures or two walls, arms, or other portions of a single structure. Such beams may be constructed in such a manner so that thickness 112 and thickness 122 are small enough to flex in response to very small forces or pressures applied to either beam 110, beam 120, or another structure attached or coupled thereto or otherwise allowing the communication of force thereto. More particularly, thickness 112 and thickness 122 may be determined in response to the type of material used to construct beam 110 or beam 120. Thickness 112 and thickness 122 may also be determined based on the level of sensitivity desired for a device used to evaluate nerve function. In one embodiment, thickness 112 and thickness 122 are similar, and may even be substantially the same thickness. For example, in one embodiment, thickness 112 and thickness 122 may each be less than 0.0001 of an inch. In another embodiment, thickness 112 and thickness 122 may each be less than 0.0005 of an inch. Testing has been conducted of dual beam structure 100 with thicknesses 112 and 122 of approximately 0.0004 of an inch that show substantially significant increases in sensitivity when used in a device for determining nerve function sensibility.

Those of skill in the art will appreciate that beams 110 and 120 may be made from a variety of materials (such as metal or polymer) depending on the intended load to be applied to such beams 110 and 120 and the desired sensitivity of beams 110 and 120. For example, in one embodiment beam 110 and beam 120 are formed of titanium. Alternatively, beam 110 and 120 may be formed of aluminum or stainless steel.

As illustrated in FIG. 1, the entire dual beam structure 100, including beams 110 and 120, may be constructed of a single material and may be machined, molded, or otherwise formed as a single component using any suitable manufacturing process. Such manufacturing capability may result in both a decrease in cost of manufacture and also an increase in robustness and product life as compared to other beam structures used to sense a load. For purposes of this application, a load shall be defined as anything acting on either beam 110, beam 120 or any other component connected, coupled, or otherwise allowing the communication of force thereto. For example, a load may be a force applied directly to beam 110 or a force applied to a probe attached to beam 110. A load may also be a pressure applied over the surface area of beam 110, portion thereof, or the tip of a probe to which beam 110 is attached.

Interconnect 130 may be any connection between beam 110 and beam 120. For example, in the illustrated embodiment, interconnect 130 is a sidewall of a single machined piece of metal that connects planar surfaces of such metal that form beam 110 and beam 120. Although interconnect 130 is illustrated as being proximate to the end of dual beam structure 100 and therefore the end of beam 110 and 120, interconnect 130 may alternatively be located elsewhere along the interior planar surfaces or edges of beam 110 and beam 120. Although illustrated as a single sidewall, interconnect 130 may include one or more interconnecting elements or surfaces between beam 110 and beam 120. Also, interconnect 130 may be deemed to include other portions of dual beam structure 100. For example, as illustrated in FIG. 1, beams 110 and 120 are further connected towards the middle by the main portion of dual beam structure 100 illustrated as base 125. Interconnect 130 is configured to be connected to a probe through aperture 140 in a manner such that a force applied to the probe is transmitted through interconnect 130 to the substantially planar walls of beams 110 and 120 in a direction transverse to the plane of the substantially planar walls.

Base 125 is a portion of dual beam structure 100 used to mount dual beam structure 100 to other portions of a sensory device. Base 125 may include aperture 150, a plurality of apertures 160, and an aperture 170. Apertures 160 may include holes used to enclose guide members to allow the lateral movement of dual beam structure 100 along a sensory device such as sensory device 200 described below relative to FIG. 1. Apertures 160 may also be machined to allow adjustment in the lateral movement of dual beam structure 100 through the use of screws or other appropriate adjustable fasteners or guide members. For example, in the dual beam structure 100 illustrated in FIG. 1, the central aperture 160 may be utilized to enclose a smooth guide member along which dual beam structure 100 may travel laterally. Exterior apertures 160 may be threaded holes to allow the adjustment of dual beam structure 100 along such smooth guide member. The threaded nature of such holes may also allow the lateral position of dual beam structure 100 to be fixed, clamped, or otherwise held in a particular desired lateral position. Aperture 150 may be utilized to attach other components of a sensory device and/or connect dual beam structure 100 to an additional dual beam structure utilizing a spring or other element to induce additional force in order to maintain a lateral position of dual beam structure 100.

Dual beam structure 100 may be generally referred to as a load sensing cell. Beam 110 and beam 120 may be substantially planar walls that are in substantially parallel relation to each other. Interconnect 130 and the side of base 125 proximate to beams 110 and 120 may form a pair of interconnect sidewalls, each of which is connected to and extends between beam 110 and beam 120. Thus, sensors 180 may be strain gauges connected to one or more of beams 110 and 120 in a manner that changes resistance, produces signals, or otherwise changes the properties of beams 110 or 120 in response to a bending of beams 110 or 120 in directions transverse to their planes. Thus, the load sensing cell that is dual beam structure 100 may be configured such that bending loads applied to the load sensing cell and in a direction substantially transverse to the planes of the substantially planar walls forming beams 110 and 120 are detectable. Dual beam structure 100 may include a support member such as base 125 that is configured to engage a support structure in a manner that enables the load sensing cell to be mounted on the support structure in canti-lever fashion.

Dual beam structure 100 also includes one or more sensors 180. Sensors 180 may be disposed on beam 110 and/or beam 120. In one embodiment, sensors 180 are strain gauges that detect the bending or flexing of beam 110 or beam 120. However, sensors 180 may be any suitable sensors whether electrical, electromechanical, optical, molecular, or any other type of sensor suitable for detecting a change in the properties or characteristics of beam 110 or beam 120 that may be indicative of the flexing, bending, or other changes in beams 110 and 120. Although described as separate sensors 180, sensors 180 may not be separate sensors or devices and may instead be resistive elements formed as part of dual beam structure 100. For example, the electrical properties of a particular region of dual beam structure 100 may be changed through the doping or deposition of additional chemicals or elements or molecular sized particles to create resistive devices out of the portions of dual beam structure 100 themselves.

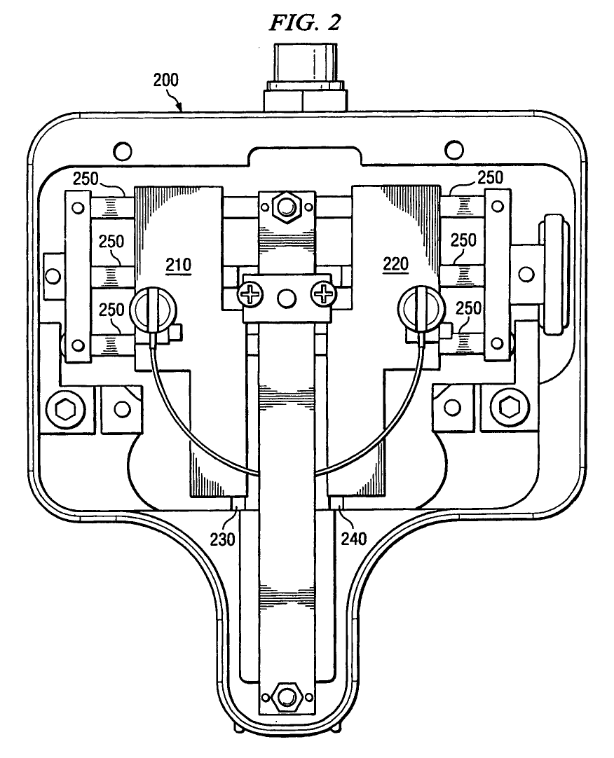

FIG. 2 illustrates one embodiment of a sensory device 200 implemented according to the teachings of the present invention and utilizing a dual beam structure such as the one previously described relative to FIG. 1. More particularly, sensory device 200 may include two dual beam structures, namely dual beam structure 210 and dual beam structure 220. Each of such dual beam structures 210 and 220 may include a hole such as aperture 140 previously described relative to FIG. 1 for the introduction of a probe. Thus, dual beam structure 220 may include two probes, each of which is connected to dual beam structure 210 and dual beam structure 220 at probe locations 230 and 240 respectively.

Sensory device 200 may also include guide members 250. As previously described relative to FIG. 1, guide members 250 may be smooth guide members along which dual beam structures 210 and 220 may travel laterally in order to configure probe locations 230 and 240 at a particular distance apart. Thus, the movement of dual beam structures 210 and 220 along guide members 250 serves to establish a distance between two probes secured at probe locations 230 and 240. As previously discussed, guide members 250 may include smooth or threaded guide members such that smooth guide members may ensure that dual beam structures 210 and 220 remain substantially parallel throughout their lateral movement. Similarly, threaded guide members 250 may be utilized to adjust the lateral position of guide members 250 in a gradual or otherwise controllable manner. Further, threaded guide members 250 may be utilized to ensure that the lateral location of dual beam structures 210 and 220, once adjusted, remain in the same position. Setting and maintaining a specific distance between the location of two probes in contact with human tissue may be useful in certain sensory tests that may be conducted using certain embodiments of a device such as sensory device 200.

Although all of the components and portions of sensory device 200 are not explained herein in exact detail, both the illustrations of FIG. 1 through 3 and the prior disclosure of U.S. Pat. No. 5,027,828 are sufficient to instruct one of ordinary skill in the art as to the implementation and use of the present invention. Further, although exact machine drawings were utilized to prepare FIGS. 1 through 3, it is important to understand that various embodiments of the present invention may be implemented and practiced without utilizing the exact structures illustrated therein.

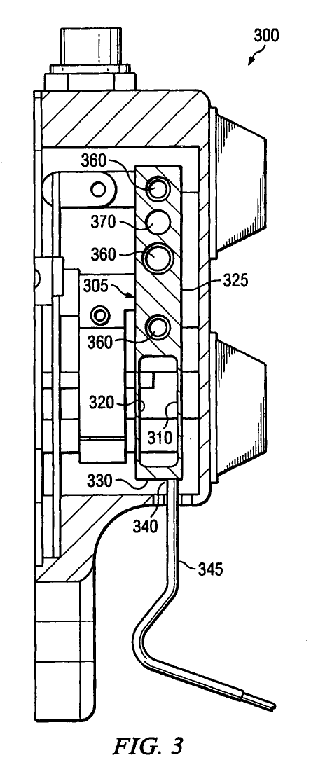

FIG. 3 illustrates one embodiment of a sensory device 300 implemented according to the teachings of the present invention. More particularly, FIG. 3 illustrates a side view of sensory device 300 that shows a dual beam structure 305 including a beam 310 and a beam 320. A probe 345 is coupled to dual beam structure 305 through an aperture 340. Dual beam structure 305 includes various additional apertures as previously described, such as a plurality of apertures 360, and an aperture 370, which form holes in a base 325. An interconnect 330 is used to connect beam 310 and beam 320.

The use of interconnect 330 is important because it allows for a decreased thickness of beams 310 and 320. Without interconnect 330, beams 310 and 320 would not have enough robustness and would be vulnerable to deformation and other damage impacting its ability to correctly sense a load applied to the end of probe 345. Indeed, without interconnect 330, it is likely that a single beam structure would need to be utilized with such single beam structure having a greater thickness than either beam 310 or beam 320. However, using the dual beam structure 305 of sensory device 300 and interconnect 330 as a portion thereof, the thickness of beams 310 and 320 may be reduced, resulting in greater sensitivity to a load applied to the end of probe 345 that will result in the flexing of beams 310 and 320.

Although probe 345 is illustrated as a curved prong, probe 345 may be any suitable prong, pin, needle, beam, button, or any other suitable component to which pressure or a force may be applied. In one embodiment, probe 345 may be manufactured as an integral part of a single machined material with dual beam structure 305 such that probe 345 is merely an extension of one or more surfaces of dual beam structure 305.

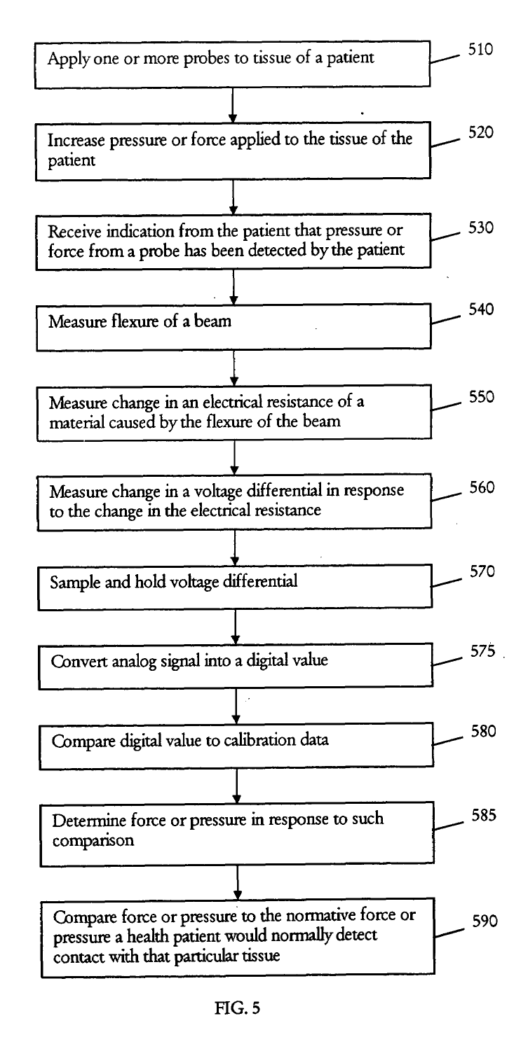

FIG. 4 illustrates one embodiment of a circuit implemented according to the teachings of the present invention that detects changes in a beam such as beams 110 and 120 of FIG. 1 such as the flexing, bending, resistance, or other measurable changes in such beams. More particularly, circuit 400 includes sensors 410, voltage generator 420, differential amplifier 430, sample-and-hold circuit 440, analog-to-digital converter 450, and processor 460. Sensors 410 may include sensors such as sensors 180 described relative to FIG. 1.

In one embodiment, sensors 410 includes resistive elements arranged in a wheatstone bridge configuration as illustrated relative to resistive elements S1, S2, S3, and S4. Such resistive elements, for example, may be part of one or more strain gauges deployed on dual beam structure 100 or may instead be resistance inherent in the material of dual beam structure 100. Voltage generator 420 is a voltage source utilized to apply a voltage signal to sensors 410. For example, generator 420 may provide a voltage signal between the junctions of resistive elements S1 and S2 and S3 and S4.

In such a manner, a voltage output at each of the junctions between resistive elements S1 and S3 and resistive elements S2 and S4 may be compared utilizing differential amplifier 430. The output of differential amplifier 430 may be sampled at particular intervals by sample and hold circuit 440 and held. The output of sample-and-hold circuit 440 is representative of the differential voltage applied across the input terminals of differential amplifier 430, which is in turn representative of the change in resistance across resistive elements S1, S2, S3, and S4, which is in turn representative of the degree to which a beam such as beam 110 of FIG. 1 may have been flexed or bent or otherwise changed.

The output of sample-and-hold circuit for 40 is in turn converted from an analog signal to a digital signal that remains indicative of the initial flexing of a beam, such as beam 110 of FIG. 1. Such digital value may then be compared by processor 460 to calibration data stored in memory that is not shown in order to determine a level of force or pressure applied to the beam, such as beam 110 of FIG. 1 or a probe or other element. Processor 460 may then compare the force or pressure applied to a beam, such as beam 110 of FIG. 1, at the time at which a patient may detect such force or pressure. Such amount of force or pressure may then be compared to normative data associated with the expected level at which a healthy patient could be expected to detect force or pressure applied to particular tissue of such patient.

FIG. 5 illustrates one embodiment of a method for determining nerve function response according to the teachings of the present invention. In step 510, one or more probes are applied to the tissue of a patient. In step 520, the pressure or force applied to the tissue of the patient is increased. In step 530, an indication is received from the patient that pressure or force from a probe has been detected by the patient. In step 540, the flexure of a beam is measured. In step 550, a change in an electrical resistance of a material caused by the flexure of the beam is measured. In step 560, a change in a voltage differential is measured in response to the change in the electrical resistance. In step 570, the voltage differential is sampled and held. In step 575, the analog signal is converted into a digital value. In step 580, the digital value is compared to calibration data. In one embodiment, the calibration data is stored in a central data repository. Alternatively, the calibration data is stored in a sensory device. In step 585, a force or pressure is determined in response to such comparison. In step 590, the force or pressure is compared to the normative force or pressure a health patient would normally detect contact with that particular tissue. Although not illustrated in FIG. 4 or FIG. 5, the differential voltage signal received as an output from sensors such as sensors 180 or sensor 410 may be amplified or filtered as appropriate to receive a waveform suitable for processing by the remainder of, for example, circuit 400 of FIG. 4.

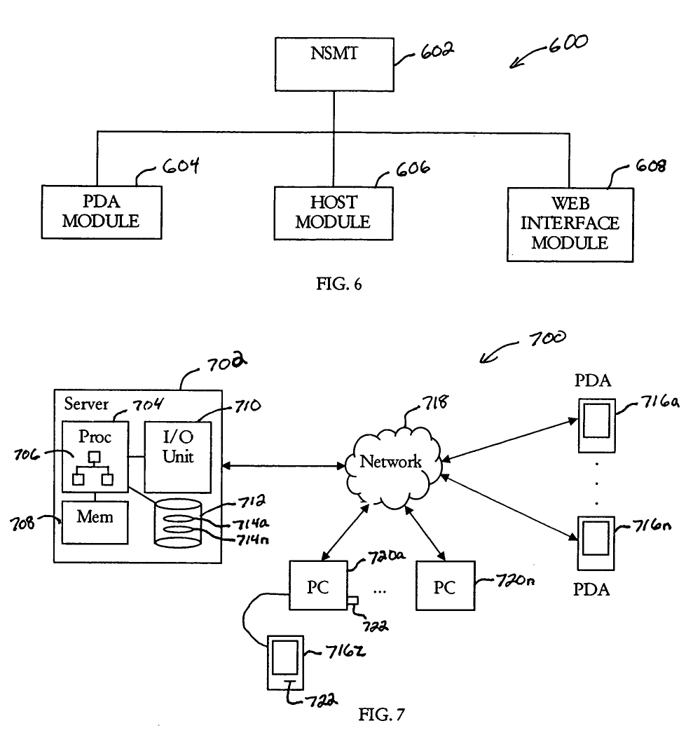

FIG. 6 is a block diagram of an exemplary configuration of a software architecture 600 for collecting and storing neurosensory data in accordance with the principles of the present invention. Neurosensory motor testing software 602 may include three software modules, a PDA module 604, host module 606, and web interface module 608. Each of these modules 604, 606, and 608 may be executed by three separate processors, including a PDA, host computer and web server, respectively (see, FIG. 7). The PDA and host software may interface via a removable flash memory card or other memory type that is used by the PDA for performing neurosensory testing. The memory card may be used to transfer patient and test data between the PDA and host. Alternatively, the data may be communicated between the PDA and host via a wire or wireless connection, as understood in the art.

The PDA module 606 may interface with the sensory device 300 of FIG. 3. The PDA module 606 controls and captures data from the sensory device 300. The PDA module 606 may perform the functions of (i) test setup, (ii) test conduct, (ii) test data storage for later upload to a host computer, and (iv) new patients addition. For the test setup function, a particular patient record may be accessed so that past test results may be viewed and new tests may be added to the patient record. For the test conduct, as the sensory device 300 is used to measure a patient's nerve functions, the PDA module 606 may read and store the measurements. In one embodiment, the measurements may be stored in association with the patient's records from which the nerve function measurements are being taken. Alternatively, a patient identifier may be associated with the test data so that the test data may be properly stored when uploaded to the host computer. The storage of the test data may also be marked as being new, not uploaded, and/or not synchronized. New patients may be added to the PDA using the PDA module 606. In one embodiment, the PDA module 606 may provide a user interface for a user to enter the patient information directly into the PDA. Alternatively, the PDA module 606 may communicate with the host module 606 and download or otherwise synchronize with the host computer to load new patient records into the PDA.

In addition to the PDA being able to test patients, the PDA module 606 may enable a user to look up, sort, and/or generate statistics of one or more patients. Historical information for a patient may be looked up and presented to a user of the PDA in tabular or graphical formats, for example. In addition, the PDA module 606 may aggregate statistics of multiple patients having a common injury or other relation (e.g., age). The aggregated statistics may be displayed to the user in a tabular or graphical format. For example, the PDA module 606 may enable a user to look up all users with a similar injury to an ulna nerve and generate a graph showing sensory recovery over time. Such generalized information may be valuable to medical professional professionals and patients seeking to determine typical recovery times of certain injuries.

Further, the PDA module 606 may enable a user to calibrate the sensory device 300 by stepping a person through a number of steps to use calibration equipment, such as a device configured to apply calibrated pressure to one or more probes of the sensory device 300. In operation, the PDA module 606 may be set into a read mode for reading output signals in response to a calibrated pressure being applied to the sensory device 300. The output signals may be a continuous stream of signals from a sample and hold circuit within the sensory device 300 or a signal indicative of the maximum force measured by the sensory device 300. Based on the measurements from the sensory device 300, the PDA module 606 may enable the user to apply an offset to cause the PDA module 606 to account for any difference between the calibration equipment and the readings by the sensory device 300. The offset may be stored by the PDA module 606 to offset measurements during patient testing. The offset may also be read by the host module 606 to monitor operation of sensory devices 300 over time.

The host module 606 is utilized to mage a patient test database and provide capabilities to process test data and produce detailed and historic test reports for medical professionals to review. The host module 606 may be configured to provide a user interface, such as a graphical user interface (GUI), for a user to perform various operations. The host module 606 may be executed on a personal computer (PC). In one embodiment, the host module 606 may enable a user to upload the neurosensory test data collected by the PDA module 606. The host module 606 may synchronize a host database with the patients currently stored on the PDA. For example, if information of a new patient is entered into the host database via the host module 606, new patient information may be downloaded to the PDA automatically or manually. For example, at the start of each day, a medical professional (e.g., physical therapist) may utilized the host module 606 to establish the patients coming in for testing that day and the host module 606 may download the records of the patients to the PDA. The host module 606 may store the neurosensory test data in a database or other data repository locally or remotely. Further, the host module 606 may be utilized to produce reports of individual patients or aggregate data of multiple patients in the same or similar manner as described with respect to the PDA module 606. It should be understood that the host module 606 may be HIPAA compliant and aggregate patient data without disclosing information specific to any patient.

The web interface module 608 may provide for one or more central databases. In one embodiment, one database may operate as an authorization database. The authorization database may be updated to specify which test units are authorized for continued use. As shown in TABLE I below, the authorization database may include parameters, including Authorized PDA's, Serial No., User Name, User ID, and User Password. Other parameters associated with the PDA's or authorized users may be included in the authorization database. A second database may be a patient test database that is updated from sensory units that are used by medical professionals on patients. The second database may include a number of different non-test parameters, including Patient Name, Patient No., and Injury. Neurosensory test information, such as test date, measurement, and notes, may be stored in the database. The measurement may be the maximum pressure measurement taken from the patient during the neurosensory test. In one embodiment, sensitivity of the sensory device ranges between 0.2 and 100 grams per square millimeter (g/mm²) for pressure, 2 mm-20 mm for distance, and sensitivity (i.e., accuracy) is 0.01 g/mm². If a pressure measurement is above 100 g/mm², it is determined that nerve fibers are dead and the sensory device may store or print out, “no one point static” or “no two point static touch,” for example. It should be understood that the sensitivity pressure ranges using the sensory device 300 is due to the strain gage bridge being split across two thin beams, as more fully described hereinabove. Other test information, such as the precise location on the patient's body of the test, may be stored in the database. Because the test database includes measurement data taken over a period of time, a doctor or other medical professional can plot the results over time on a graph and determine the progress of the patient.

TABLE I
Exemplary Authorization Database

| Authorized | | | | User |
|---|---|---|---|---|
| PDA's | Ser. No. | User Name | User ID | Password |
| PDA1 | 97234T742 | Jeffrey Samuels | JSamuels | JeffTSam |
| PDA2 | 09782F234 | Richard Capon | RCapon | RCap73 |
| PDA3 | 84726P413 | Marty Sousa | MSousa | XiSousa |

TABLE II
Exemplary Patient Test Data

| | | | | Meas. | | Meas. | | |
|---|---|---|---|---|---|---|---|---|
| Patient Name | Patient No. | Injury | Test Date | g/mm² | Test Date | g/mm² | . . . | Notes |
| Susan Segraves | 027434 | Ulna Nerve | Oct. 14, 2006 | 47.87 | Oct. 21, 2006 | 46.28 | . . . | Acute |
| | | | | | | | | sensitivity |
| Pablo Hilton | 908723 | Radial Nerve | Oct. 14, 2006 | 34.72 | Oct. 21, 2006 | 31.74 | . . . | Redness |
| Darin Collins | 972344 | Ulna Nerve | Oct. 15, 2006 | 83.95 | Oct. 22, 2006 | 83.14 | . . . | Scaring |
| | | | | | | | | developing |
| Greg Belair | 741722 | brachial plexus | Oct. 15, 2006 | 64.31 | Oct. 22, 2006 | 57.74 | . . . | Flexibility |
| | | | | | | | | increasing |

The web interface module 608 may additionally provide for updating software in the host module 606 and PDA module 604. In addition to provide for database management, the web interface module 608 may be utilized to enable a user to view test data and generate reports from the test data. The web interface module 608 may enable a user to perform statistical analysis on the test data in an aggregate manner compliant with the Health Insurance Portability and Accountability Act of 1996 (HIPAA) rules.

FIG. 7 is a block diagram of an architecture of an exemplary portable test device system 700. The system 700 includes a server 702 that includes a processor 704 that executes software 706. A memory 708, input/output (I/O) Unit 710, and storage unit 712 may be in communication with the processor 704. The memory 708 may be utilized to store test data and software 706 while being executed. The I/O unit 710 may be utilized to communicate information internal and external from the I/O unit 710. The storage unit 712 may store one or more databases 714a-714n (collectively 714) or other data repositories of neurosensory test data collected from neurosensory test devices (e.g., sensory device 300). PDA's 716a-716n (collectively 716) that are used for neurosensory testing may be in communication with a network 718, such as the Internet or an intranet within a healthcare facility (e.g., hospital, for communicating neurosensory test data collected from testing patients with the sensory device, for example. In communicating over the network 718, the PDA's 716 may include transceivers (not shown) for wirelessly communicating over the network 718. Alternatively, as illustrated, a PDA 716z may include a memory card 722 that inserts into the PDA 716z during operation of the PDA 716z during neurosensory testing.

Personal computers (PCs) 720a-720n (collectively 720) may be in communication with the network 718. The PCs 720 may operate as host computers that are in communication with the network 718. When a medical professional desires to upload the test data taken from patients, the memory card 722 may be removed from the PDA 716z and inserted in the PC 720a or adapter connected thereto. Data stored on the memory card 722 may be read by software, such as the host module 606, and uploaded onto a database being stored on the PC 720a. Alternatively and/or additionally, the test data may be uploaded to the database 714 at the server 702. The PCs 720 may be utilized to interact with the databases 714 to access data of particular patients, generate statistical analysis, and view reports of aggregated test data. In one embodiment, the server 720 is a personal computing device configured to operate as a server. The software 706 may operate the web interface module 608, the host PCs 720 may operate the host module 606, and the PDA's 716 may operate the PDA module 604.

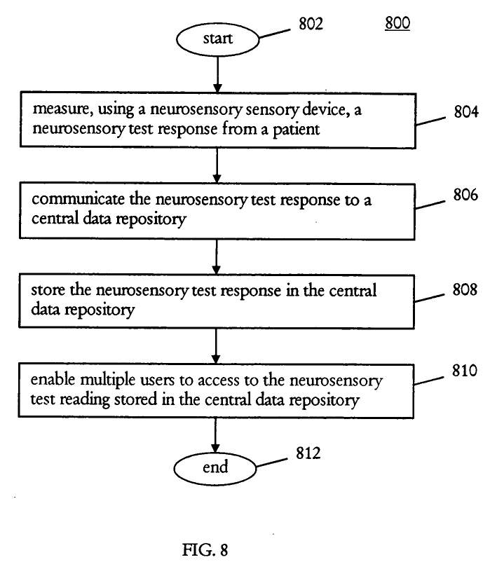

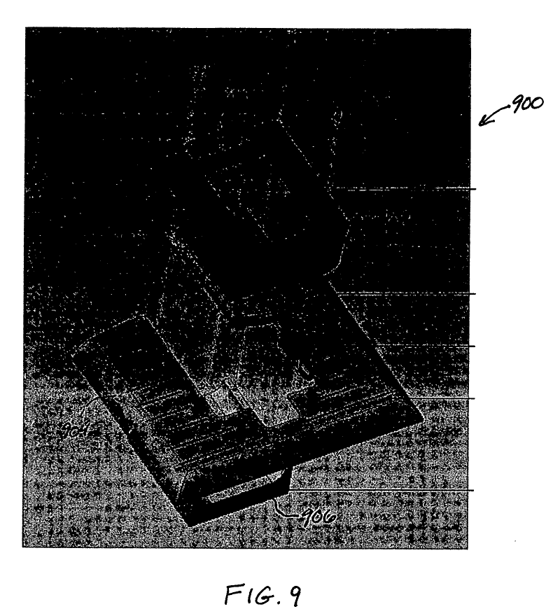

FIG. 8 is a flow diagram 800 of an exemplary process for measuring and storing neurosensory test data. The process 800 starts at step 802. At step 804, a neurosensory test response is measured from a patient using a handheld sensory device. The neurosensory test response may be an indication by a patient of feeling two probes, if using a multi-probe device, being pressed against his or her skin. Indication of a response be made in many ways, including moving a body part or making a vocal response to feeling the two probes. At step 806, the neurosensory test response is communicated to a central data repository (e.g., database). In one embodiment, the neurosensory test response is made from the handheld sensory device via a network. Alternatively, the neurosensory test response may be stored in a memory device, uploaded into a host computer, and communicated to a server or other computing system, via a network or other communication link that manages the central data repository. At step 808, the neurosensory test response may be stored in the central data repository. Multiple users may be provided access to the neurosensory test response stored in the central data repository at step 810. The process ends at step 812.

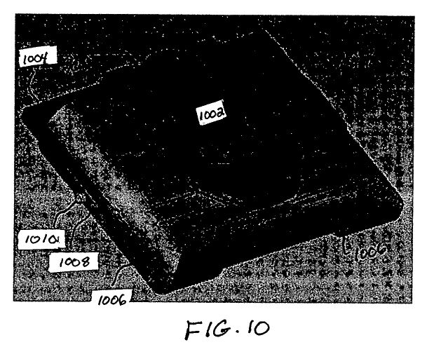

FIG. 9 is an illustration of an exploded view of an exemplary housing 900 for enclosing a handheld computing device 902, such as a PDA, for collecting data from a sensory device used to perform neurosensory testing on patients. The housing 900 may include a top cover 904 configured to support a wide range of handheld computing devices and enable a user to access user interface features of the devices. The housing 900 may enclose sampling electronics (not shown), positioned on one or more printed circuit boards or otherwise, that are used to collect the neurosensory test data that is sensed by the sensory device (e.g., sensory device 300). The housing cover 904 may further include an input and/or output port for connecting the sensory device to the sampling electronics. By enclosing the sampling electronics within the housing 900, the user of the handheld computing device 902 may simply interface with the handheld computing device 902 and not have to transport multiple devices (i.e., the handheld computing device 902 and electronics) or make connections between the two. In addition, by including the handheld computing device 902 and electronics, which are connected to one another within the housing 900, the overall testing system is “cleaner” in that one unit may be carried around to perform the neurosensory testing. The housing cover 904 may include a handle (not shown) or other carrying mechanism to make it easier for a user of the test system to carry.

As further illustrated in FIG. 9, the housing 900 may include a cradle 906 for receiving the handheld computing device 902. The cradle 906 may be shaped and sized to receive and support a wide range of handheld computer configurations. A bezel 908 is configured to maintain the handheld computing device 902 in the cradle 906 by connecting to either the top cover 904 or cradle 906 via the top cover 904 by connection members 910 (e.g., screws). The bezel 908 defines an opening 912 that allows full functionality of the handheld computing device 902 by a user. It should be understood that “full functionality” of a handheld computing device means that the following functions of the handheld computing device can be utilized without interference from the housing: (i) access to any buttons or switches on the front of the handheld computing device 902, (ii) access to any stylus, which is generally accessed at the top of a handheld computing device to control the handheld computing device 902, and/or (iii) visibility of the screen of the handheld computing device. As handheld computing device capabilities and case designs change often (at least once a year), the ability to accommodate newer design handheld computing devices with minimal impact to the housing 900. By having a modular housing design with a large enough cavity for a wide range of handheld computing device sizes to fit within the cradle 906, handheld computing devices with new designs are accommodated. The handheld computing device 902 is held captive with the bezel 908 that exposes necessary portions of the handheld computing device body, such as the flash memory card slot, the stylus holder, and the front panel buttons while hiding the internal wiring to the handheld computing device 902.

As described, the housing 900 may be configured to support a wide range of handheld computing device configurations. Through the use of the cradle 906 and bezel 908, virtually any Windows CE based handheld computing device may be accommodated without change to the case design. To protect the electronics and handheld computing device 902, the top cover and other surrounding structure (e.g., cradle 906 and bezel 908) may be formed of plastic, aluminum, or any other material that protects the internal data acquisition hardware, power supply and handheld computing device 902 to perform sensory tests.

FIG. 10 is an illustration of the exemplary housing 900 of FIG. 9 in a working configuration. As shown, the handheld computing device 902 is configured below the bezel 908 and held in by the cradle (not shown). The configuration of the housing 900 enables a user to access a user interface 1002 (e.g., electronic display, keypad, or other user interactive mechanism) of the handheld computing device 902. As shown, an exemplary handle 1004 may be connected to a base 1006 via a securing member 1008 (e.g., screw). A hinge or slide member 1010 may also be included so that the housing can be more easily carried by a user. Sensing electronics (not shown) may be located under the housing cover 904 and be connected to an input connector (not shown).

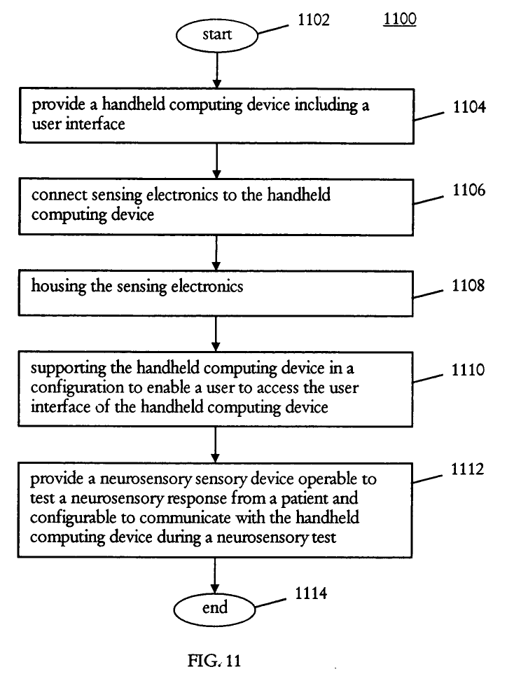

FIG. 11 is a flow diagram of an exemplary process 1100 for configuring an apparatus for testing a neurosensory response from a patient. The method starts at step 1102. At step 1104, a handheld computing device including a user interface is provided. Sensing electronics are connected to the handheld computing device at step 1106. The sensing electronics may be housed at step 1108. At step 1110, the handheld computing device may be supported in a configuration to enable a user to access the user interface of the handheld computing device. At step 1112, a neurosensory sensory device, operable to test a neurosensory response from a patient, may be configurable to communicate with the handheld computing device during a neurosensory test. The process 1100 ends at step 1114.

Utilization of the sensory device and other principles of the present invention provide the ability to measure dynamic changes (i.e., one and two-point moving touch), which was heretofore not possible. The sensory device permits the evaluation of nerve regeneration because the one-point moving touch recovers before one-point static touch and two-point moving touch recovers before two-point static touch.

Although particular embodiments of the present invention have been explained in detail, it should be understood that various changes, substitutions, and alterations can be made to such embodiments without departing from the spirit and scope of the present invention as defined solely by the following claims.

What is claimed:

1. A system for managing neurosensory test information, said system comprising:
a computing system operating on a network;
a storage unit in communication with said computing system;
a data repository stored in said storage unit, said data repository configured to store neurosensory test data read by a neurosensory test apparatus; and
means for communicating the neurosensory test data to said data repository.

2. The system according to claim 1, wherein the neurosensory test apparatus is a handheld device.

3. The system according to claim 1, wherein the neurosensory test apparatus includes a dual beam structure configured to sense neurosensory responses from the patient in response to a pressure being applied to a tissue of the patient during a neurosensory test.

4. The system according to claim 3, wherein the neurosensory test apparatus is configured to store a maximum pressure applied to the patient during a neurosensory test.

5. The system according to claim 1, wherein said data repository is a database configured to be remotely accessed over the network by multiple users.

6. The system according to claim 1, wherein said data repository is configured to store names of patients, dates of neurosensory tests, and neurosensory test readings.

7. The system according to claim 1, further comprising software, executed by said computing system, and configured to operate a user interface to enable users to access data stored in said data repository.

8. The system according to claim 7, wherein the user interface enables a user to view neurosensory data stored in said data repository for a particular patient.

9. The system according to claim 7, wherein said software is further configured to provide access to the neurosensory data in a HIPAA compliant manner.

10. The system according to claim 7, wherein said software is further configured to aggregate data stored in said data repository.

11. The system according to claim 10, wherein said software is further configured to generate statistics from the aggregated data.

12. A method for managing neurosensory test information, said method comprising:
measuring a neurosensory test response from a patient;
communicating the neurosensory test response to a central data repository;
storing the neurosensory test response in the central data repository; and
enabling multiple users to access to the neurosensory test reading stored in the central data repository.

13. The method according to claim 12, wherein measuring the neurosensory test response includes applying a force to a patient until the patient senses the force and measuring the force at which the patient senses the force.

14. The method according to claim 13, wherein applying the force includes applying a force with two probes and wherein measuring the force at which the patient senses the force is performed when the patient senses the force from both probes.

15. The method according to claim 12, wherein storing the neurosensory test response includes storing a maximum neurosensory test response.

16. The method according to claim 12, wherein storing the neurosensory test response includes storing the test response in association with a name of the patient and date of the neurosensory test.

17. The method according to claim 12, wherein enabling multiple users to access the neurosensory test reading includes providing a user interface configured to enable users to access data stored in the central data repository.

18. The method according to claim 17, wherein providing the user interface enables a user to view neurosensory data stored in said data repository for the patient.

19. The method according to claim 18, wherein providing the user interface enables a user to access to the neurosensory data in a HIPAA compliant manner.

20. The method according to claim 18, wherein providing the user interface enables a user to aggregate data stored in the central data repository.

21. The method according to claim 20, wherein providing the user interface enables a user to generate statistics from the aggregated data.
